(19) United States
(12) Patent Application Publication
(10) Pub. No.: US 2022/0112553 A1
(43) Pub. Date: Apr. 14, 2022

(54) METHODS FOR SINGLE-MOLECULE ANALYSIS OF LINEARIZED POLYNUCLEOTIDES

(71) Applicants: Massachusetts Institute of Technology, Cambridge, MA (US); The Broad Institute, Inc., Cambridge, MA (US)

(72) Inventors: Edward S. Boyden, Cambridge, MA (US); Fei Chen, Cambridge, MA (US); Nikita Obidin, Cambridge, MA (US); Andrew Colin Payne, Cambridge, MA (US)

(73) Assignees: Massachusetts Institute of Technology, Cambridge, MA (US); The Broad Institute, Inc., Cambridge, MA (US)

(21) Appl. No.: 17/500,060

(22) Filed: Oct. 13, 2021

Related U.S. Application Data
(60) Provisional application No. 63/090,754, filed on Oct. 13, 2020.

(51) Int. Cl. C12Q 1/6874; C12Q 1/6806

(52) U.S. Cl. CPC C12Q 1/6874; C12Q 1/6806

(57) ABSTRACT

Methods for single-molecule analysis of structure and sequence of linearized polynucleotides are provided.

### RELATED APPLICATIONS

This application claims benefit under 35 U.S.C. § 119(e) of U.S. Provisional application Ser. No. 63/090,754 filed Oct. 13, 2020, the disclosure of which is incorporated by reference herein in its entirety.

### GOVERNMENT SUPPORT

This invention was made with government support under UF1NS107697, 1R01EB024261, 1R01DA045549, and 1R01MH114031 awarded by the National Institutes of Health. The government has certain rights in the invention.

### FIELD OF THE INVENTION

The invention relates, in part, to methods of novel linearized sequence and structure determination of polynucleotides.

### BACKGROUND OF THE INVENTION

There is a need for methods of polynucleotide analysis at higher resolutions, longer lengths, and greater sensitivities than are currently available. Methods which analyze single molecules are inherently maximally sensitive and avoid complications, such as amplification errors, associated with methods which do not have single-molecule sensitivity [Schirmer, M. et al., Nucleic Acids Res. 43, e37 (2015)]. Moreover, single-molecule methods based on polynucleotide linearization are, in principle, capable of analyzing very long molecules, on the order of hundreds to thousands of kilobases (kb) for DNA [Kaykov A., et al., Sci. Rep. 6, 19636 (2016)].

However, current DNA linearization methods have two drawbacks that limit the genomic resolution at which the linearized DNA may be analyzed. First, linearized DNA is typically analyzed using optical microscopy, and therefore the smallest adjacent genomic features that can be uniquely distinguished is limited by the classical diffraction limit of light. That limit is about 1 kilobase (1 kb) for a fully elongated molecule and good optics, which is insufficient to resolve many genomic structural variations [He Y-S., et al., Hereditas (Beijing) 31, 771-778 (2009)]. Second, once DNA is linearized and immobilized on a surface, it cannot be subjected to efficient enzymatic reactions, because enzymatic reactions are inhibited by the solid phase [Marie R., et al., Nanoscale 10, 1376-1382 (2018)]. Although some surfaces are compatible with limited enzymatic activity, such solid-phase reactions are still inefficient compared to the liquid phase [Gupta A., et al., Microfluidics and Nanofluidics 20, doi: 10.1007/s10404-015-1685-y (2016)].

Because the most practical sequencing chemistries are based on sequential rounds of enzymatic processing [Shendure J., et al., Nature 550, 345-353 (2017)], linearization methods cannot achieve base-pair level resolution of resolved loci, thus failing to measure the most common type of genetic variation.

### SUMMARY OF THE INVENTION

According to an aspect of the invention, a method of determining a structure and sequence of a polynucleotide is provided, the method including: (a) modifying the polynucleotide with a bi-functional cross-linker molecule; (b) linearizing the polynucleotide; (c) immobilizing the linearized polynucleotide; (d) embedding the polynucleotide in a polymer material; (e) fragmenting the embedded polynucleotide; (f) physically expanding the polynucleotide fragments; (g) detecting spatial position and sequences of the expanded polynucleotide fragments; and (h) determining a structure and sequence of the polynucleotide. In certain embodiments, the modifying comprises functionalizing the polynucleotide. In some embodiments, a means for functionalizing the polynucleotide includes conjugating the polynucleotide to the bi-functional cross-linker molecule, wherein the bi-functional cross-linker molecule comprises at least one polynucleotide-reactive group and at least one material-reactive moiety. In some embodiments, the at least one polynucleotide-reactive group comprises a DNA binding domain and the at least one material-reactive moiety comprises a polymerizable domain. In certain embodiments, the DNA binding domain is an alkylating group and the polymerizable domain is an acryloyl group. In some embodiments, the DNA binding domain comprises a DNA binding protein. In some embodiments, the DNA binding protein comprises a zinc finger, TALEN, dCas9, or other inactive CRISPR associated proteins. In some embodiments, the DNA binding domain comprises one or more intercalating agents, optionally an acridine compound. In certain embodiments, a means for functionalizing the polynucleotide comprises modifying the polynucleotide. In some embodiments, the modified polynucleotide comprises one or more modified nucleotides. In certain embodiments, if one of the modified nucleotides includes an EdC modified nucleotide, the binding domain on the bi-functional crosslinker comprises an azide group and if one of the modified nucleotides includes a VdU modified nucleotide, the binding domain on the bi-functional crosslinker comprises a tetrazine group. In certain embodiments, the method is performed on a plurality of the polynucleotide. In some embodiments, functionalizing the plurality of polynucleotides comprises conjugating the polynucleotides with two or more different bi-functional cross-linkers. In some embodiments, the polynucleotide-reactive group comprises one or more of a DNA probe and a DNA-binding antibody. In some embodiments, the material-reactive moiety comprises one or more of a methacrylate and an acrylate. In certain embodiments, a means for the linearizing comprises a molecular combing method or capillary action method. In some embodiments, a means for the immobilizing includes one or more of DNA binding, DNA binding in combination with a receding meniscus method, a heat-adhesion method, a fixation method, or a capillary action method. In some embodiments, the fixation method comprises an ethanol or a formaldehyde fixation method. In certain embodiments, the linearized polynucleotide is immobilized on a solid support. In some embodiments, the solid support comprises one or more of: polystyrene, polymethylmethacrylate (PMMA), polylysine, polyhistidine, glass, silica, metal, and plastic. In some embodiments, the solid support comprises a vinyl silane surface, an aminosilane surface, or a PDMS surface. In some embodiments, the polymer material comprises a swellable polymer material. In certain embodiments, the swellable polymer material comprises an acrylamide-co-acrylate copolymer. In certain embodiments, the material comprises a non-swellable polymer material capable of conversion to a swellable polymer material, and the method also includes converting the non-swellable polymer material into a swellable polymer material. In some embodiments, the method also includes converting the non-swellable polymer material into a swellable polymer material prior to the physically expanding of the polynucleotide fragments. In some embodiments, the non-swellable polymer material comprises a non-swellable hydrogel. In some embodiments, the non-swellable hydrogel comprises one or more of an acrylamide and polyacrylate. In some embodiments, fragmenting the embedded polynucleotide comprises contacting the polymer material in which the polynucleotide is embedded with one or more of (i) a strong base and (ii) one or more DNA-cleaving enzymes. In certain embodiments, the method also includes cleaving the polymer material comprising the embedded polynucleotide from the solid support. In some embodiments, a method of the cleaving comprises contacting the polymer material with a strong base. In certain embodiments, the strong base is one or more of NaOH, KOH, LiOH, RbOH, CsOH, Ca(OH)₂, Sr(OH)₂, and BA(OH)₂. In some embodiments, the method also includes double-stranded denaturing the polynucleotide fragments embedded in the polymer material prior to the physical expansion of the polynucleotide fragments, wherein the double-stranded denaturing of the nucleotide fragments generates single-stranded polynucleotide fragments. In some embodiments, a means of the physically expanding the polynucleotide fragments comprises expanding the polymer material in which the polynucleotide fragments are embedded, wherein the expansion of the polymer material expands the polynucleotide fragments isotropically in at least a linear manner within the polymer material. In certain embodiments, the polymer material comprises a hydrogel and a means of expanding the hydrogel comprises contacting the hydrogel with an aqueous solution, optionally water. In certain embodiments, the physically expanded polynucleotide fragments are re-embedded in the same or a different polymer prior to the detecting, optionally wherein the detecting comprises DNA sequencing. In some embodiments, the re-embedding is in a non-swellable polymer. In some embodiments, the physically expanded polynucleotide fragments are not re-embedded in a polymer prior to the detecting. In certain embodiments, the method also includes passivating the expanded polymer. In certain embodiments, the detecting comprises one or both of imaging and sequencing the polynucleotide fragments. In certain embodiments, a means for the detecting comprises a method capable of capturing spatial data. In some embodiments, the means for the detecting comprises transferring the fragments from the polymer to a spatially indexed array, wherein the spatially indexed array optionally comprises a microarray or a bead array. In some embodiments, a means for detecting the transferred expanded polynucleotide fragments comprises a PCR method or a DNA sequencing method. In some embodiments, the means for the detecting comprises sectioning the expanded polymer, identifying the relative positions of the sections, recovering DNA from the sections, detecting the polynucleotide fragments, associating the detected fragments with their identified relative positions, and determining the spatial positions and sequences of the associated detected fragments. In certain embodiments, the sectioning comprises sectioning as an indexed grid. In some embodiments, a means of detecting the polynucleotide fragments comprises a PCR method or a DNA sequencing method. In some embodiments, the means for the detecting comprises microscopy. In some embodiments, the microscopy is fluorescence microscopy or transmission electron microscopy. In certain embodiments, the method also includes detectably labeling the polynucleotide fragments. In some embodiments, a means for the detectably labeling comprises directly or indirectly attaching one or more detectable labels to the polynucleotide fragments. In some embodiments, the detectably labeling comprises affinity labeling, wherein optionally the affinity label comprises one or more of biotin, digoxigenin, and a hapten. In some embodiments, a means for the detectable labeling comprises contacting the polynucleotide fragments with one or more enzymes, under suitable conditions for activity of the one or more enzymes to result in detectable labeling of polynucleotide fragments. In certain embodiments, a means of indirectly attaching the detectable label to the polynucleotide fragments comprises hybridizing one or more detectably labelled DNA probes to the polynucleotide fragments. In some embodiments, the detectable label comprises a fluorescent label, a luminescent label, a radiolabel, an enzymatic label, a contrast agent, a heavy metal, or a heavy element. In some embodiments, the polymer in which the polynucleotide is embedded is not contacted with a detergent. In some embodiments, a means for the sequencing includes: (a) hybridizing one or more primer molecules to the polynucleotide fragments; (b) amplifying the polynucleotide fragments; and (c) determining sequences of the amplified polynucleotide fragments. In certain embodiments, the primer is a random sequence primer. In certain embodiments, the primer is preselected to target a locus of interest. In some embodiments, the locus of interest is associated with a disease or condition. In some embodiments, the disease or condition comprises a monogenic disorder, a chromosomal disease, a polygenic disorder. In certain embodiments, the method also includes classifying the detected spatial positions and sequences of the expanded polynucleotide fragments into one or more contiguous polynucleotide molecule. In certain embodiments, a means of the classifying comprises identifying the spatial positions of the detected polynucleotide fragments in one or more dimensions and determining a relative ordering of the detected sequences of the polynucleotide fragments within a single contiguous polynucleotide molecule, wherein the relative ordering aids in classifying the detected sequences into one or more contiguous polynucleotide molecules and identifying a structure of the one or more contiguous polynucleotide molecules. In some embodiments, the method also includes identifying the presence or absence of a structural variation in the one or more classified contiguous polynucleotide molecule, compared to a control structure. In some embodiments, the structural variation identified as present in the one or more classified contiguous polynucleotide molecules is associated with a disease or condition. In certain embodiments, the structural variation identified as present in the one or more classified contiguous polynucleotide molecules is, when presented in a cell, associated with a disease or condition. In certain embodiments, the disease or condition is a cancer. In some embodiments, the polynucleotide is obtained from a cell. In some embodiments, the cell is obtained from a subject. In certain embodiments the subject is a genetically engineered subject. In certain embodiments, the subject is a mammal, optionally is a human. In some embodiments, the cell is a cultured cell. In some embodiments, the cell is a mammalian cell. In certain embodiments the cell is a genetically engineered cell.

### BRIEF DESCRIPTION OF THE DRAWINGS

FIG. 1 shows a flowchart depicting steps in an embodiment of the method of the invention.

FIG. 2A-C shows schematic drawings illustrating steps of an embodiment of the method. FIG. 2A depicts functionalization of a polynucleotide by conjugating the polynucleotide with a bi-functional cross-linker molecule. FIG. 2B depicts linearization of a functionalized polynucleotide on a solid support. FIG. 2C depicts the formation of a polymer (e.g. hydrogel) overlay on the solid support, followed by simultaneous cleavage of the overlay from the support, fragmentation of the hydrogel-embedded polynucleotides, and physical expansion of the hydrogel. In FIG. 2C, chemical linkers are indicated with asterisks, and reporter probes are indicate with open triangles.

FIG. 3 presents a photomicrograph showing microscopic detection of lambda phage DNA prior to expansion according to a method of the invention. The lambda phage DNA was labelled for detection using fluorescence in situ hybridization (FISH).

FIG. 4 presents a photomicrograph showing microscopic detection of expanded lambda phage DNA according to a method of the invention. In this example, the lambda phage DNA was labelled for detection using FISH.

FIG. 5 presents a photomicrograph showing microscopic detection of expanded lambda phage DNA according to a method of the invention. In this example, the lambda phage DNA was labelled for detection using the enzymatic method of random primer extension.

FIG. 6 presents a photomicrograph showing an example of microscopic detection of expanded lambda phage DNA according to a method of the invention. In this example, the lambda phage DNA was labelled for detection using the enzymatic method of terminal transferase tailing.

FIG. 7 shows a graph comparing the length distribution of lambda phage DNA before (diagonal-line fill) and after (crosshatched fill) expansion; lengths are inferred from the longest dimension of spatially proximal puncta in microscopic images. Prior to expansion, the distribution exhibited a sharp peak at ˜20 μm (the length of fully extended lambda phage) with a smaller peak at ˜10 μm (half the length, caused by DNA bound to the solid support during linearization by both of its extremities [Strick et al., Progress in biophysics and molecular biology 74.1-2 (2000): 115-140]. After twofold linear expansion, lambda phage lengths increased significantly and are proportionally longer (***p<0.001, KS test).

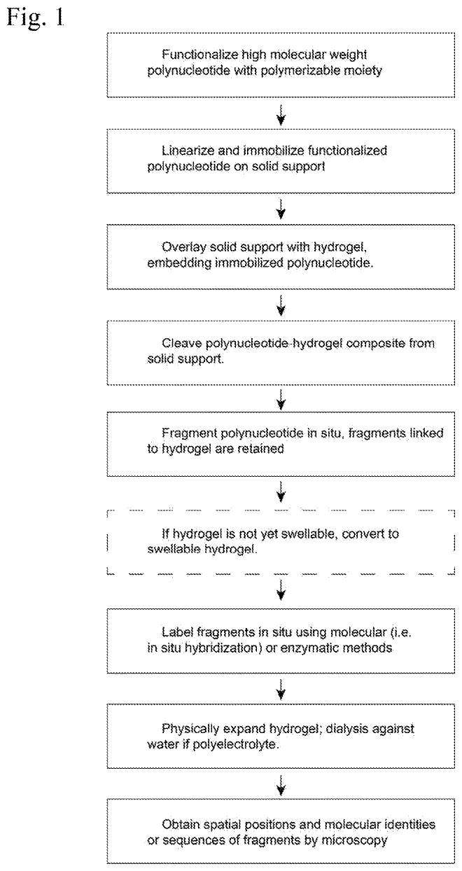

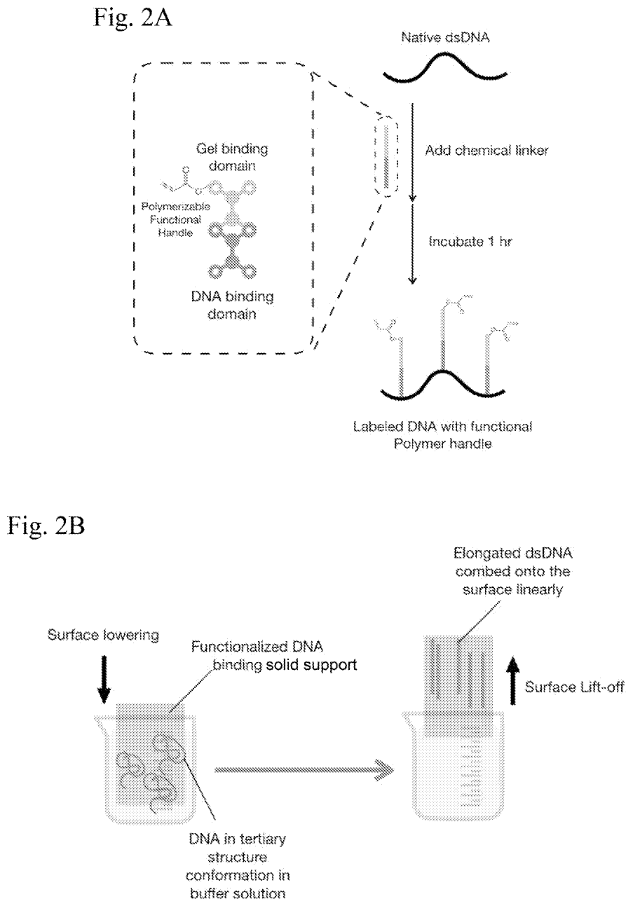

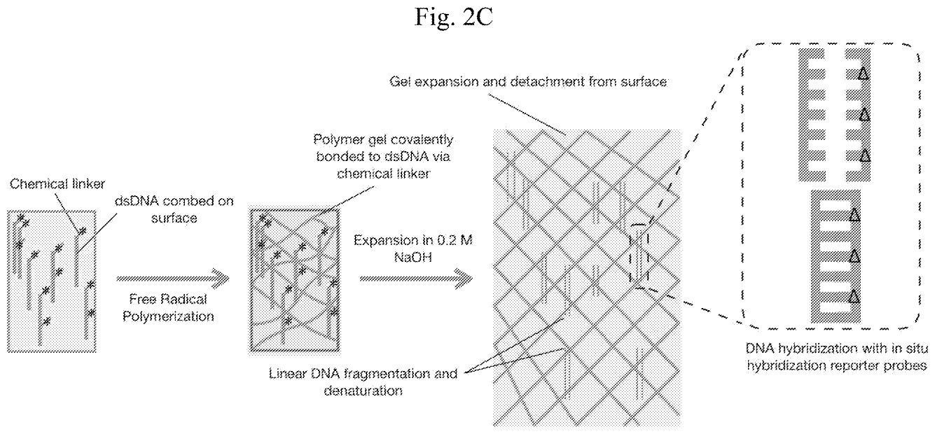

### DETAILED DESCRIPTION

Existing methods, such as next generation sequencing (NGS), require multiple rounds of enzymatic processing and cannot sequence very long polynucleotides. The present invention comprises methods for obtaining the structure and sequence of long polynucleotides.

Existing ExM methods are intended for biological specimens in a cellular or tissue context: chemically fixed biomolecules within the specimen are covalently embedded in a swellable material; the ultrastructure of the specimen is then digested; and the material is expanded, physically separating the biomolecules. Throughout that process, individual anchored biomolecules, including polynucleotides, remain fixed in their native, compact conformation. Therefore, even after expansion, individual biomolecules are localized to within a single post-expansion diffraction-limited spot. As a result, although the identity and position of individual biomolecules may be recovered, their linear structure cannot be determined beyond the length of short-read sequencing, at most 500 bp. [Chen F., et al., Nat. Methods 13, 679-684 (2016); Chen F., et al., Science 347, 543-548 (2015)]. Aspects of the invention comprise methods of preparing long polynucleotides for microscopic and enzymatic analysis below the diffraction limit of light.

The invention, in part, provides methods for obtaining a structure and a sequence of long polynucleotides. Methods of the invention comprise modifying a polynucleotide with a cross-linker bearing a reactive polymerizable moiety, which is also referred to herein as “functionalizing” the polynucleotide. The functionalized polynucleotide is linearized, immobilized, and embedded in a material. In some embodiments, the material comprises a swellable polymer material, and in certain embodiments of the invention, the material comprises a non-swellable polymer material that is capable of conversion to a swellable polymer material. Following embedding, the polynucleotide undergoes controlled fragmentation and the resulting fragments are physically expanded. In certain embodiments of methods in which the linearized, immobilized nucleotides are embedded in a non-swellable material, the method may also include converting the non-swellable polymer material into a swellable polymer material prior to the physically expanding of the polynucleotide fragments. Swelling of the material results in a physical expansion of the fragments from their positions in the material. Certain methods of the invention also include detecting the fragments using methods such as but not limited to hybridization and by enzymatic techniques, and the results of the detection provides one or both of structural and sequence information about the original polynucleotide.

Methods of the invention, in part, include preparing polynucleotides for enzymatic and microscopic analysis below the diffraction limit of light; to do this, embodiments of methods of the invention utilize a physical expansion of biomolecules in a polymer, a non-limiting example of which is a hydrogel. Unlike prior ExM methods, embodiments of the invention disclosed herein permit recovery of spatial structure and sequence of individual polynucleotide biomolecules.

Embodiments of methods of the invention permit detection of structure and sequences of unfixed and elongated polynucleotides. In certain embodiments, methods include embedding such polynucleotides in a polymer, for example but not limited to an acrylamide polymer, followed by digestion, such as but not limited to NaOH digestion, and swelling of the polymer comprising the embedded polynucleotides. In certain embodiments, methods of the invention may be used for genomic DNA detection, assembly and analysis, including determining alternations in genomic DNA. In certain embodiments, methods of the invention may be used to identify assess DNA sequences such as, but not limited to: a genomic DNA sequence from a subject; a wild-type (control) genomic DNA sequence; a genetically engineered genomic DNA sequence, a genomic DNA sequence known to be or suspected of being associated with a disease or condition. Methods of the invention can be used to identify genomic DNA sequences and structures as well as differences in genomic DNA obtained from different sources. As a non-limiting example, methods of the invention may be used to compare structure and/or sequence of normal (e.g. control) genomic DNA to structure and/or sequence of genomic DNA obtained from a subject who has, or is suspected of having a disease or condition. Differences between the determined genomic DNAs may assist in identifying a genomic variation or abnormality associated with the subject's disease or condition. Methods of the invention are able to provide genomic information beyond that obtainable from assessment of spatial localization of RNA molecules, or DNA molecules in unextended conformations.

### Polynucleotides

The term “nucleotide” as used herein includes a phosphoric ester of nucleoside—the basic structural unit of nucleic acids (DNA or RNA). The terms “polynucleotide” and “nucleic acid” refer to a polymer comprising multiple nucleotide monomers and may be used interchangeably herein. A polynucleotide may be either single stranded, or double stranded with each strand having a 5′ end and a 3′ end. The end regions of a stretch of nucleic acid may be referred herein to as the 5′ terminus and the 3′ terminus, respectively. A nucleotide in a polynucleotide may be a natural nucleotide (deoxyribonucleotides A, T, C, or G for DNA, and ribonucleotides A, U, C, G for RNA), or may be a “modified nucleotide”, which as used herein refers to a non-natural or derivatized nucleotide base or nucleotide otherwise chemically or biochemically modified. In some embodiments of the invention, one or more modified nucleotides are incorporated into a polynucleotide by, for example, chemical synthesis, or may result from contacting a polynucleotide with a reagent capable of modifying a nucleotide during or after isolation of the polynucleotide from a source or during methods of the invention. Such modified nucleotides may confer additional desirable properties absent or lacking in the natural nucleotides; and polynucleotides comprising modified nucleotides may be used in the compositions and methods of the invention. As used herein, a “modified polynucleotide” refers to a polynucleotide comprising at least one modified nucleotide. In some embodiments, a modified polynucleotide may comprise one, two, three, four, five, or more modified nucleotides, non-limiting examples of which are: an EdC modified nucleotide (5-ethynyl-2′-deoxycytidine) and a VdU modified nucleotide (5-vinyl-2′-deoxyuridine).

A polynucleotide may be DNA (including but not limited to cDNA or genomic DNA), RNA, or hybrid polymers (e.g., DNA/ RNA). The terms “polynucleotide” and “nucleic acid” do not refer to any particular length of polymer. Polynucleotides used in embodiments of methods of the invention may be at least 1, 2, 3, 4, 5, 10, 20, 30, 40, 50, 60, 70, 80, 90, 100, 200, 500, 1000, 2000, or 5000 kb or more in length. The term “sequence,” used herein in reference to a polynucleotide, refers to a contiguous series of nucleotides that are joined by covalent bonds, such as phosphodiester bonds. The term “structure” as used herein in reference to a polynucleotide, refers to overall sequence organization of a polynucleotide, including “structural variations” such as insertions, deletions, repeats, and rearrangements. A polynucleotide may be chemically or biochemically synthesized, or may be isolated from a subject, cell, tissue, or other source or sample that comprises, or is believed to comprise, nucleic acid sequences including, but not limited to, cDNA, mRNA, and genomic DNA. A polynucleotide assessed using a method of the invention may be a chromosome molecule, a fragment of a chromosome molecule (that is additionally fragmented while in the polymer); etc.

Methods describe herein can be used to assess polynucleotides of various lengths, including, but not limited to polynucleotides significantly longer than those that can be accurately assessed using prior methods. Embodiments of certain methods of the invention result in higher read length and lower error rates than prior methods, see for example, Kaykov, Atanas, et al. (2016) Scientific reports 6.1): 1-9.

Parameters useful to evaluate effectiveness and efficiency of identification of polynucleotide sequences using and embodiment of the present invention or a previous method include, but are not limited to read length and error rate. Previous methods based on short-read fluorescence sequencing achieved error rates on the order of 0.1% per base but are generally limited to at most 500 nt read lengths. ExSeq is a non-limiting example of this prior sequencing means. Previous methods based on e.g. single-molecule real time sequencing (for example but not limited to: PacBio) or nanopore (for example but not limited to: Oxford Nanopore) can achieve routine read lengths of ˜30,000 nt (PacBio) to 200,000 (Nanopore). However, the error rates for these technologies are very high (˜5% per base). In contrast, embodiments of methods of the invention can achieve higher read lengths than current long read technologies (up to 10 Mb), while achieving error rates comparable to short-read fluorescence sequencing (0.1%). Certain non-limiting examples of different polynucleotides and lengths that may be that may be identified and sequence using an embodiment of the invention are: 500 nt (a non-limiting example of which is: a single fragment); 500-10,000 nt (non-limiting examples of which are: highly sheared chromosomal DNA, some viral genomes); 10,000-100,000 nt (non-limiting examples of which are: lightly sheared chromosomal DNA, some viral genomes); 100,000-1,000,000 nt (non-limiting examples of which are: chromosomal DNA handled to minimize shearing, some viral and bacterial genomes); and 1,000,000-10,000,000 (a non-limiting example of which is: chromosomal DNA handled in agarose as in Kaykov et al).

### Functionalization

To prepare polynucleotides for linearization, embedding, expansion, detection, and analysis, a polynucleotide may be functionalized, which, as used herein, comprises being modified with a bi-functional cross-linker molecule, or by modifying the polynucleotide. As used herein, a “bi-functional cross-linker” molecule is a small molecule comprising at least one polynucleotide-reactive group and at least one material-reactive moiety, and capable of attaching to a target nucleic acid and to the material of the expansion gel. In some embodiments, attaching the small molecule to the target nucleic acid may be accomplished by a chemically reactive group or groups capable of covalently binding the target nucleic acid. As used herein, the term “attach” or “attached” refers to both covalent interactions and noncovalent interactions. In certain embodiments of the invention, covalent attachment may be used, but generally, all that is required is that the nucleic acids remain attached to the target material. In aspects of the invention, a bi-functional cross linker (“Label-X”) bearing an alkylating moiety and an acryloyl moiety is produced by coupling the small molecule Acryloyl-X (ThermoFisher Scientific, Waltham, Mass.) to the small molecule Label-IT® Amine (Mirus Bio, Madison, Wis.) as described [Chen F., et al., Nat. Methods 13, 679-684 (2016)].] A plurality of polynucleotides may he functionalized with two or more different bi-functional cross-linker molecules.

In some embodiments, a polynucleotide-reactive group comprises a DNA binding domain, such as an alkylating group, an azide, or a tetrazine; in other embodiments, a DNA binding domain is a DNA binding protein comprising a zinc finger, TALEN, dCas9, or other inactive CRISPR associated protein, or a DNA-binding antibody. In other embodiments, a DNA binding domain comprises DNA probes binding via hybridization. A DNA binding domain may also comprise one or more intercalating agents, including acridine compounds. In certain embodiments of methods of the invention, a material-reactive moiety comprises a polymerizable domain, such as methacrylates and acrylates. For example, in some embodiments, the DNA binding domain may be an alkylating group and the polymerizable domain may be an acryloyl group.

In certain embodiments of the invention, the polynucleotide to be functionalized comprises one or more modified nucleotides. In certain embodiments, one of the modified nucleotides includes an EdC modified nucleotide and the binding domain on the bi-functional cross linker comprises an azide group. In some embodiments, one of the modified nucleotides includes a VdU modified nucleotide and the binding domain on the bi-functional crosslinker comprises a tetrazine group.

### Linearization and Immobilization

The functionalized polynucleotide is linearized and immobilized on a solid support. As used herein, a “solid support” means one or more of a polystyrene, a polymethylmethacrylate (PMMA), a polylysine, a polyhistidine, a glass, a silica, a metal, a plastic, a vinyl silane, an aminosilane, or a PDMS surface (see, for example, U.S. Pat. No. 5,840,862, which is incorporated by reference herein in its entirety). Linearization methods include molecular combing (a combination of DNA binding by an extremity to the surface in tandem with the action of a receding meniscus [Bensimon A., et al., Science 265, 2096-2098 (1994)]), and capillary action. Immobilization methods include nonspecific adhesion due to heat, or a fixation method such as ethanol or formaldehyde fixation.

### Embedding

Linearized functionalized DNA is embedded in a swellable polymer, or in a polymer that can be chemically converted into a swellable polymer. For example, the polymer may be acrylamide; acrylamide can later be converted into an acrylamide-co-acrylate copolymer after treatment with a strong base such as sodium hydroxide, which can then swell after dialysis with water. Other polymers such as polyacrylate could be considered. The polymer may be cast in a thin overlay over the solid support, and may bind to the solid support when the support itself has reactive groups that can participate in free radical polymerization, or otherwise nonspecifically bind the gel, as is the case, for example, with a vinyl silane surface and aminosilane surface respectively.

As used herein, the term “swellable polymer material” generally refers to a material that expands when contacted with a liquid, such as water or other solvent [Wassie A., et al., Nat. Methods 16, 33-41 (2019); and U.S. Pat. No. 10,059,990 in relation to swellable and non-swellable materials, each publication is incorporated by reference herein in its entirety.]

The swellable material may uniformly expand in three dimensions. Additionally or alternatively, the material is transparent such that, upon expansion, light can pass through the sample. In some embodiments, the swellable polymer material is a swellable polymer or hydrogel. In one embodiment, the swellable polymer is formed in situ from precursors thereof: for example, one or more polymerizable materials, monomers or oligomers may be used, such as monomers selected from the group consisting of water-soluble groups containing a polymerizable ethylenically unsaturated group. Monomers or oligomers may comprise one or more substituted or unsubstituted methacrylates, acrylates, acrylamides, methacrylamides, vinylalcohols, vinylamines, allylamines, allylalcohols, including divinylic crosslinkers thereof (e.g., N,N-alkylene bisacrylamides). Precursors may also comprise polymerization initiators and crosslinkers.

In some embodiments, a swellable polymer is an acrylamide-co-acrylate copolymer, polyacrylate, or polyacrylamide, or co-polymers or cross-linked co-polymers thereof. Alternatively or additionally, the swellable polymer may be formed in situ by chemically cross-linking water-soluble oligomers or polymers. Thus, the invention envisions adding precursors, such as water-soluble precursors, of the swellable polymer to the sample and rendering the precursors swellable in situ.

As used herein, the term “non-swellable polymer material” material comprises a polymer material capable of conversion to a swellable polymer material, including a non-swellable hydrogel comprising one or more of an acrylamide and polyacrylate [Ueda H., et al., Nat. Rev. Neurosci. 21, 61-79 (2020)]. In some embodiments of the invention, the polymer is not a polyacrylade polymer.

In some embodiments of the invention, the polynucleotides are embedded in a non-swellable polymer material and the non-swellable material is converted into a swellable polymer material prior to the physically expansion of the polynucleotide fragments. In a non-limiting example, a non-swellable polymer comprises acrylamide, which contacted with a strong based such as sodium hydroxide and thereby converted into an acrylamide-co-acrylate copolymer, which is a swellable polymer that will swell with dialysis with water.

### Surface Detachment, Fragmentation, and Polymer Material Conversion

Once the linearized polynucleotide has been embedded and immobilized in a polymer overlay, methods of the invention undertake simultaneous steps of surface detachment, polynucleotide fragmentation, and hydrogel conversion. As used herein, “surface detachment” means that the polymer overlay is cleaved from the solid support such that the embedded DNA remains in the gel phase rather than adhering to the support. Means for cleaving the polymer overlay from the solid support include exposing the support-DNA-overlay system to a strong base, which, in the case of a silane-type surface cleaves siloxane bonds to glass, freeing the polymer overlay. In some embodiments, the strong base is one or more of NaOH, KOH, LiOH, RbOH, CsOH, Ca(OH)₂, Sr(OH)₂, and BA(OH)₂.

As used herein, “polynucleotide fragmentation” or “fragmenting an embedded polynucleotide” means that polynucleotides bound to the gel are fragmented in place prior to the physical expansion of the polymer, in which the polynucleotides are embedded, and fragments bound to the polymer are retained and double-stranded denaturing of the nucleotide fragments generates single-stranded polynucleotide fragments. In some embodiments of the invention prior to the physical expansion of the polymer material, the physical structures of the embedded polynucleotides are disrupted. Physical disruption of the polynucleotides, which may be referred to herein as “physical disruption”, may result from one or more of a physical, chemical, biochemical, and enzymatic digestion, disruption, and breakup of the polynucleotides so they will not resist expansion when the polymer in which they are embedded is expanded. Some embodiments of the invention include use of a protease enzyme to disrupt the polynucleotide(s). It will be understood that certain embodiments of the invention methods are capable of disrupting the embedded polynucleotides without altering the structure of the polymer material. In some embodiments of the invention, a means of disrupting a polynucleotide embedded in a polymer is selected so the method does not significantly alter the polymer material in which the polynucleotide is embedded, but physically disrupts the polynucleotide to an extent sufficient to permit expansion of the polynucleotide when the polymer material in which it is embedded is expanded.

Non-limiting examples of means for fragmenting an embedded polynucleotide include incubating the support-DNA-overlay system with a strong base such as sodium hydroxide, especially when abasic sites are present in the DNA [Maxam A. M. and Gilbert W., Proc. Natl. Acad. Sci. U.S.A. 74, 560-564 (1977)]; incubation with DNA-cleaving enzymes (e.g. restriction enzymes, transposase); and chemical methods. Fragmentation may be controlled by modulating the number of abasic sites when using a strong base, or by choosing more- or less-specific restriction enzymes.

### Labelling

Linearized DNA fragments bound to the gel are “labelled” or “tagged” with a detectable label. As used herein, the term “detectable label” means a label or tag that is chemically bound to the polynucleotide, or to a component thereof, through covalent, hydrogen, or ionic bonding, and is detected using microscopy or one or more other means of detection. A detectable label may be selective for a specific target (e.g., a biomarker or class of molecule), as may be accomplished with an antibody or other target specific binder, or the detectable label may be an affinity label, including one or more of biotin, digoxigenin, and a hapten. In some embodiments, a detectable label comprises a visible component, as is typical of a dye or fluorescent molecule, a luminescent label, a radiolabel, an enzymatic label, a contrast agent, a heavy metal, or a heavy element such as bromine or iodine, or metals such as gold, osmium, rhenium, etc.; however any signaling means used by the label is also contemplated. A fluorescently labeled polynucleotide, for example, is a polynucleotide labeled through techniques such as, but not limited to, immunofluorescence, immunohistochemical, or immunocytochemical staining to assist in microscopic analysis. In some embodiments, the detectable label is a probe, antibody, and/or fluorescent dye, wherein the antibody and/or fluorescent dye further comprises a physical, biological, or chemical anchor or moiety that attaches or crosslinks the sample to the composition, polymer (e.g., hydrogel), or other swellable material. The detectable label may be attached to the nucleic acid adaptor, and in some embodiments, more than one label may be used. For example, each label may have a particular or distinguishable fluorescent property, e.g., distinguishable excitation and emission wavelengths. Further, each label may have a different target-specific binder that is selective for a specific and distinguishable target in, or component of the sample. In other embodiments, the detectable label is indirectly attached to the polynucleotide by means of hybridizing one or more detectably labelled probes to the polynucleotide fragments, such as fluorescently labelled DNA probes, or probes bearing detectable makers such as haptens, may label DNA by hybridization.

A “probe” generally refers to a nucleic acid molecule or a sequence complementary therewith, used to detect the presence of at least a portion of a target sequence. The detection may be carried out by identification of hybridization complexes between the probe and the assayed target sequence. The probe may be attached to a solid support or to a detectable label. Probe(s) are generally single-stranded, and may be at least 10, 20, 50, 100, 200, 500, 1 kb, 2 kb, 5 kb, or 10 kb or more nucleotides in length. The particular properties of a probe will depend upon the particular use(s) for which it is intended, and are within the competence of one of ordinary skill in the art to determine. Generally, a probe will hybridize to at least a portion of the target sequence under conditions of high-stringency hybridization.

In other embodiments, enzymatic methods for detectable labeling are used, including contacting the polynucleotide fragments with one or more enzymes, under suitable conditions for activity of the one or more enzymes to result in detectable labeling of polynucleotide fragments.

### Polymer and Nucleotide Expansion

The polymer (a non-limiting example of which is a hydrogel) within which linearized polynucleotide fragments are embedded is isotropically expanded. In some embodiments, a solvent or liquid is added to the complex and the solvent or liquid is absorbed by the swellable material and causes swelling. For example, if the mechanism of expansion is the polyelectrolyte effect, the gel may be dialyzed against water or an aqueous solution to expand. In one embodiment, the addition of water allows the embedded sample to expand at least 3, 4, 5, or more times its original size in three dimensions. Thus, the sample may be increased 100-fold or more in volume. The labelled, linearized polynucleotide, having been fragmented, therefore expands isotropically along with the gel in at least a linear manner.

As used herein the terms “passivating” or “passivation” refer to a process for rendering a polymer material less reactive with components contained within the polymer material. In some embodiments of the invention, passivation of a polymer comprising a polynucleotide is used to reduce and/or prevent unwanted downstream enzymatic reactions. A non-limiting example of passivation of a polymer material is functionalizing the polymer material with one or more chemical reagents to neutralize charges within the polymer material. In some embodiments of the invention, a swellable polymer containing expanded nucleotide fragments is not passivated. Certain embodiments of the invention an expanded swellable polymer comprising polynucleotide fragments may be re-embedded in a non-swellable or in a swellable polymer prior to detection of the polynucleotide fragments. A re-embedded swellable polymer may be partially or completely degraded chemically, provided the polynucleotide fragments in the polymer either remain anchored or are transferred to the non-swellable polymer. In some embodiments of the invention, non-charged polymer chemistries may be used to avoid charge passivation. In certain embodiments of the invention, the physically expanded polymer and polynucleotide fragments are not re-embedded in a polymer prior to being detected.

In a non-limiting example of a passivation procedure the swellable gel can be passivated by contacting the polymer containing the polynucleotide(s) with 1-Ethyl-3-(3-dimethylaminopropyl)carbodiimide (EDC) and N-Hydroxysuccinimide (NHS) to covalently react ethanolamine to the carboxylic groups. First, the polymer containing the polynucleotide is re incubated with 2M Ethanolamine hydrochloride, 150 mM EDC, 150 mM NHS, and 100 mM 2-(N-morpholino)ethanesulfonic acid (MES) buffer ph 6.5 for 2 hours. Next, the polymer containing the polynucleotides is incubated with 2M Ethanolamine hydrochloride and 62 mM Sodium borate (SB) buffer at pH 8.5 for 40 minutes. Additional passivation methods are routinely used in the art and are suitable for use in conjunction with embodiments of methods of the invention.

### Detecting Structure and Sequence

Methods of the invention allow detection of spatial structures and sequences of the expanded polynucleotide fragments using microscopic and enzymatic detection methods. The signal from individual molecules may be spatially punctate due to the fragmentation step. However, these puncta will be spatially proximal, allowing the overall length of the linearized DNA to be inferred based on the dimension in which spatial proximity is highest. Thus, the structure of the polynucleotide may be inferred over distances up to the entire length of the linearized molecule, which may be hundreds to thousands of kilobases.

As used herein, “detecting” means using one or both of an imaging method and a sequencing method to identify the spatial position and sequences of polynucleotides. Imaging methods include but are not limited to FISH, fluorescence microscopy, confocal microscopy, epi-fluorescence microscopy, spinning disk microscopy, two-photon microscopy, light sheet microscopy, total internal reflection (TIRF) microscopy, super resolution microscopy, or transmission electron microscopy. Enzymatic detection methods include but are not limited to random primer extension, terminal transferase tailing, padlock probe rolling circle amplification [Larsson, C., et al. Nat. Methods 1(3): 227-232 (2004)], in situ PCR [Hodson, R., et al. Appl. Environ. Microbiol. 4074-4082 (1995)], horseradish peroxidase tyramide signal amplification [Schonhuber, W., et al. Appl. Environ. Microbiol. 3268-3273 (1997)], luciferase-catalyzed pyrophosphate chemiluminescence [Nyren, P., et al. Anal. Biochem. 208:171-175 (1993)], or other PCR-based or DNA sequencing methods [Stahl, P. L., et al. Science 353.6294: 78-82 (2016); Rodrigues, S. G., et al., Science 363.6434: 1463-1467 (2019)]. As used herein, “spatial position” refers to the location of one polynucleotide or polynucleotide fragment relative to the location of another polynucleotide or polynucleotide fragment. Certain embodiments of methods of the invention are useful to determine relative positions of one or more fragments generated from a single linear molecule. For example, fragments generated from a linear molecule, such as but not limited to: a whole chromosome; a piece of a chromosome, which is then further fragmented; a viral genome, etc., can be identified and sequenced using methods of the invention which can be used to identify the relative positions of the fragments in the original linear molecule. Thus, embodiments of methods of the invention can be used to disarticulate a polynucleotide molecule in to fragments in a controlled manner and then to identify the sequences and relative positions of the resulting fragments in the linear/extended conformation.

An additional aspect of the invention is that the DNA is transferred from a solid phase support to a quasi-liquid-phase hydrogel, which is >99% liquid phase. Because many enzymatic reactions are inefficient on solid phase supports, linearized and immobilized DNA is rarely analyzed in this manner. In specific circumstances, a judicious choice of surface and linearization technique permits the possibility of enzymatic reaction, including but not limited to the steps of (a) hybridizing one or more primer molecules to the polynucleotide fragments; (b) amplifying the polynucleotide fragments; and (c) determining sequences of the amplified polynucleotide fragments. Transfer of the DNA from the solid phase support to the polymer (a non-limiting example of which is a hydrogel) in certain embodiments of the invention, allows efficient enzymatic reactions to proceed on elongated DNA. Some embodiments of methods of the invention are used to perform multiple rounds of enzymatic sequencing. Thus, individual sub-sequences making up an entire polynucleotide may be spatially localized along the length polynucleotide, allowing structural variation to be spatially resolved at resolutions down to single base pairs. In some embodiments of the invention, a primer is preselected to target a locus of interest and in some embodiments a primer used in a method of the invention is a random-sequence primer.

The term “primer” or “priming sequence” as used herein, refers to an oligonucleotide capable of acting as a point of initiation of DNA synthesis under suitable conditions for synthesis of a primer extension product complementary to a nucleic acid strand. In some embodiments, the initiation of DNA synthesis occurs in the presence of four different nucleoside triphosphates and an agent for extension (non-limiting examples of which are: a DNA polymerase and a reverse transcriptase) in an appropriate buffer and at a suitable temperature. A primer may be a single-stranded DNA. The appropriate length of a primer depends on the intended use of the primer but typically ranges from 10 to 50 nucleotides, such as from 15-35 nucleotides. Short primer molecules generally require lower temperatures to form sufficiently stable hybrid complexes with the template. A primer need not reflect the exact sequence of the template nucleic acid, but must be sufficiently complementary to hybridize with the template. The design and use of suitable primers for the amplification of a given target sequence is well known in the art and described in, for example, the literature cited herein.

The term “sequencing” as used herein refers to one or more of various methods used to determine the order of constituents in a biopolymer, in this case, a polynucleotide used in methods of the invention or sub-sequences resulting from enzymatic reactions performed on polynucleotides of the invention. Suitable sequencing techniques that may be used with the instant invention includes the traditional chain termination Sanger method, as well as the so-called next-generation (high throughput) sequencing available from a number of commercial sources, such as massively parallel signature sequencing (or MPSS, by Lynx Therapeutics/Solexa/Illumina), polony sequencing (Life Technologies), pyrosequencing or “454 sequencing” (454 Life Sciences/ Roche Diagnostics), sequencing by ligation (SOLiD sequencing, by Applied Biosystems/ Life Technologies), sequencing by synthesis (Solexa/Illumina), DNA nanoball sequencing, heliscope sequencing (Helicos Biosciences), ion semiconductor or Ion Torrent sequencing (Ion Torrent Systems Inc./Life Technologies), and single-molecule real-time (SMRT) sequencing (Pacific Bio), etc. Numerous other sequencing and high-throughput sequencing methods are also suitable for use to sequence polynucleotides in methods of the invention, including but not limited to: nanopore DNA sequencing, sequencing by hybridization, sequencing with mass spectrometry, microfluidic Sanger sequencing, transmission electron microscopy DNA sequencing, RNAP sequencing, and in vitro virus high-throughput sequencing, etc.

In some embodiments of methods of the invention, a means for detecting may comprise transferring the fragments from the polymer to a spatially indexed array, wherein the spatially indexed array optionally comprises a microarray or a bead array in order to capture spatial data. In some embodiments methods of the invention, a means for the detecting comprises one or more of: sectioning the expanded gel, identifying the relative positions of the sections, recovering DNA from the sections, detecting the polynucleotide fragments, associating the detected fragments with their identified relative positions, and determining the spatial positions and sequences of the associated detected fragments [Kebschull, J. M., et al., Neuron 91.5: 975-987 (2016)]. In some embodiments, the expanded gel may be sectioned as an indexed grid. A non-limiting example of an embodiment of the invention utilizing an indexed grid includes sectioning the polymer into pieces using e.g. a knife, keeping track of (indexing) the relative positions of the sections. These sections are then processed independently (i.e. through DNA retrieval and conventional sequencing), and the relative positions of sequenced molecules in one section relative to other sections can then be reconstructed.

### Analysis

Certain embodiments of methods of the invention may be used to analyze structure spatial organization, and sequence of one or more loci of interest that may be known or may be suspected of being associated with a disease or condition. Some embodiments of methods of the invention can be used to identify loci associated with a disease or condition. Non-limiting examples of diseases and conditions that can be assessed using embodiments of the invention are: monogenic disorders such as but not limited to: sickle cell anemia, hemophilia, cystic fibrosis, Tay Sachs disease, Huntington's disease, and fragile X syndrome; chromosomal disorders such as but not limited to: Down syndrome and Turner syndrome; polygenic disorders such as but not limited to Alzheimer's disease, heart disease, cancers, and diabetes, etc. Certain embodiments of methods of the invention can also be used to assess polynucleotides, sequences, and structures associated with gene structural disorders, non-limiting examples of which are: gene deletions, gene insertions, gene rearrangements, gene duplications, repeat expansions; and cancers, etc.

In conjunction with methods of the invention, art-known methods may be used to assess relative sequence identity between two nucleic acid sequences. For example, two sequences may be aligned for optimal comparison purposes, and the nucleic acids at corresponding positions can be compared. When a position in one sequence is occupied by the nucleic acid in the corresponding position in the other sequence, then the molecules have identity/similarity at that position. The percent identity or percent similarity between the two sequences is a function of the number of identical positions shared by the sequences (i.e., % identity or % similarity=number of identical positions/total number of positions×100). Such an alignment may be performed using any one of a number of well-known computer algorithms designed and used in the art for such a purpose. It will be understood that a variant polynucleotide sequence may be shorter or longer than their parent polynucleotide sequence, respectively. The term “identity” as used herein in reference to comparisons between sequences may also be referred to as “homology”.

In aspects of the invention, the detected spatial structures and sequences of the expanded polynucleotide fragments are classified into one or more contiguous polynucleotide molecules. In some embodiments of the invention, classifying comprises identifying spatial positions of detected polynucleotide fragments in one or more dimensions and determining a relative ordering of detected sequences of polynucleotide fragments within a single contiguous polynucleotide molecule, wherein the relative ordering aids in classifying the detected sequences into one or more contiguous polynucleotide molecules and identifying a structure of the one or more contiguous polynucleotide molecules. Some embodiments of methods of the invention may be used to determine a relative ordering of DNA fragment lengths, which is then used to assemble detected sequences by scaffolding shorter DNA reads. [Stankova, H., et al. Plant Biotech. J. 14: 1523-1531 (2016); Jain, M. et al. Nat. Biotech. doi:10.1038/nbt.4060 (2018)]. In other embodiments, the presence or absence of a structural variation in one or more classified contiguous polynucleotide molecules is identified compared to a control structure, and may be identified as being present in one or more classified contiguous polynucleotide molecules associated with a disease or condition. Certain embodiments of methods of the invention can be used to identify one or more structural variations on elongated DNA [Gad et al., J. Med. Genet. 38: 388-392 (2001); Cheeseman, K., et al. Human Mutation 33 (6): 998-1009 (2012)].

### Subjects and Cells

The term “subject” may refer to human or non-human animals, including mammals and non-mammals, vertebrates and invertebrates, and may also be any multicellular organi sin or single-celled organism such as a eukaryotic (including plants and algae) or prokaryotic organism, archaeon, microorganisms (e.g., bacteria, archaea, fungi, protists, viruses), and aquatic plankton. A subject may be considered to be a normal subject or may be a subject known to have or suspected of having a disease or condition. In some embodiments, an organism is a genetically modified organism. As used herein the term “genetically modified” is used interchangeably with the term “genetically engineered”.

Cells, tissues, or other sources or samples may include a single cell, a variety of cells, or organelles. It will be understood that a cell sample comprises a plurality of cells. As used herein, the term “plurality” means more than one. In some instances, a plurality of cells is at least 1, 10, 100, 1,000, 10,000, 100,000, 500,000, 1,000,000, 5,000,000, or more cells. A plurality of cells from which polynucleotides are isolated for use in methods of the invention may be a population of cells. A plurality of cells may include cells that are of the same cell type. In some embodiments, a cell from which polynucleotides are isolated for use in methods of the invention is a healthy normal cell, which is not known to have a disease, disorder, or abnormal condition. In some embodiments, a plurality of cells from which polynucleotides are isolated for use in methods of the invention includes cells having a known or suspected disease or condition or other abnormality, for example, a cell obtained from a subject diagnosed as having a disorder, disease, or condition, including, but not limited to a degenerative cell, a neurological disease-bearing cell, a cell model of a disease or condition, an injured cell, etc. In some embodiments, a cell is an abnormal cell obtained from cell culture, a cell line known to include a disorder, disease, or condition. Non-limiting examples of diseases or conditions include monogenic disorders, such as sickle cell anemia, hemophilia, cystic fibrosis, Tay Sachs disease, Huntington's disease, and fragile X syndrome; chromosomal disorders, such as Down syndrome and Turner syndrome; polygenic disorders such as Alzheimer's disease, heart disease, diabetes, etc.; structural disorders such as deletions, insertions, and repeat expansions; and cancers. In some embodiments of the invention, a plurality of cells is a mixed population of cells, meaning all cells are not of the same cell type. Cells may be obtained from any organ or tissue of interest, including but not limited to: skin, lung, cartilage, brain, CNS, PNS, breast, blood, blood vessel (e.g., artery or vein), fat, pancreas, liver, muscle, gastrointestinal tract, heart, bladder, kidney, urethra, and prostate gland. In some embodiments, a cell from which polynucleotides are isolated for use in methods of the invention is a control cell. In various embodiments, cells from which polynucleotides are isolated for use in methods of the invention may be genetically modified or not genetically modified.

A cell from which polynucleotides are obtained for use in methods of the invention may be obtained from a biological sample obtained directly from a subject. Non-limiting examples of biological samples are samples of: blood, saliva, lymph, cerebrospinal fluid, vitreous humor, aqueous humor, mucous, tissue, surgical specimen, biopsy specimen, tissue explant, organ culture, biological fluid or any other tissue or cell preparation, or fraction or derivative thereof or isolated therefrom, etc. In some embodiments of the invention, polynucleotides may be obtained from primary cells, cell lines, freshly isolated cells or tissues, frozen cells or tissues, paraffin embedded cells or tissues, fixed cells or tissues, and/or laser dissected cells or tissues. In some embodiments, a sample from which polynucleotides are isolated for use in methods of the invention is a control sample. Polynucleotides may be isolated from a subject, cell, or other source according to methods known in the art. A cell or subject from which a polynucleotide obtained for use in an embodiment of a method of the invention may be a genetically engineered cell or subject, respectively.

### EXAMPLES

### Example 1

### Expansion and Detection of Lambda Phage DNA

Single molecules of linearized lambda phage (λ-phage) DNA were expanded, labeled, and imaged.

### Materials and Methods

PNA preparation: Functionalization, Linearization, Immobilization, and Embedding

To functionalize the DNA, a bifunctional crosslinker (“Label-X”) bearing an alkylating moiety and an acryloyl moiety was produced by coupling the small molecule Acryloyl-X (ThermoFisher Scientific, Waltham, Mass.) to the small molecule Label-IT Amine (Minis Bio LLC) as described [Chen F., et al., Nat. Methods 13, 679-684 (2016)], The Label-X crosslinker was then reacted with full-length λ-phage DNA (New England Biolabs, Ipswich, Mass.; 5 μg λ-phage DNA and 1:20 Label-X in Buffer A (Mints Bio, Madison, Wis.)) at room temperature for one hour with agitation, followed by storage at −20° C.

To linearize labeled full-length λ-phage DNA, immobilize it on a solid support, and embed it, Label-X-modified DNA was diluted to 10 pM in 150 mM MES buffer, pH 5.5, and was then elongated and immobilized on a hydrophobic vinyl silane modified coverslip (Biosurfaces, Inc., Ashland, Mass.) using the molecular combing technique as described [Kaykov A., et al., Sci. Rep. 6, 19636 (2016)]. Monomer solution (1× PBS, 4% (w/w) acrylamide, 0.2% (w/w) N,N′-Methylenebisacrylamide) was mixed fresh. 0.1% (w/v) of ammonium persulfate (APS) and tetramethylethylenediamine (TEMED) were added to the monomer solution up to 0.2% (w/w) each and immediately brought into contact with the surface-immobilized DNA. The solution was sandwiched between a second glass coverslip and incubated in a humidified chamber at 37° C. for one hour, resulting in an acrylamide surface overlay, approximately 50 μm thick and adherent to the vinyl silane surface.

### Surface Detachment, DNA Fragmentation, and Polymer Conversion

The sample was treated with a strong base to simultaneously (1) cleave the overlay from the surface while retaining DNA in the gel phase, (2) fragment the DNA, (3) convert the gel to a sweilable polymer, and (4) denature the DNA. Specifically, the overlay-coverslip sample was incubated in 0.2 M NaOH overnight. First, treatment with a strong base cleaved the surface same bonds, reversing DNA surface immobilization and thereby detaching the DNA-polymer (e.g., DNA-hydrogel) composite from the glass surface [Rosch L., et al., Ullmann's Encyclopedia of Industrial Chemistry, doi: 10.1002/14356007.a24_021 (2000)]. Second, treatment with a strong base permitted controlled fragmentation of the DNA, because when the DNA was modified by Label-X, a majority of sites were functionalized as described elsewhere herein, but a minority of sites were damaged and rendered abasic [Kondo N., et al., J. Nucleic Acids, 1-7 (2010)], making them available for efficient cleavage by a strong base [Maxam A. M. and Gilbert W., Proc. Natl. Acad. Sci. U.S.A., 74, 560-564 (1977)]. The ratio of polymerizable DNA adducts and abasic DNA sites may in principle be modulated by doping Label-X with unmodified Label-IT, thus controlling the degree of fragmentation. Third, the MOH treatment denatured DNA [Wang X., et al., Environ. Health Toxicol., 29, e2014007 (2014)], making it accessible for downstream hybridization or enzymatic reactions which require single-stranded DNA. Finally, treatment with a strong base converted a portion of acrylamide hydrogel side chains into acrylate, which, as validated [Chang J.-B., et al., Nat. Methods, 14, 593-599 (2017)], caused the gel to expand isotropically when dialyzed with water or low salt content buffer.

### DNA Labelling

To facilitate microscopic detection of DNA, fluorescence in situ hybridization (FISH) was performed on the DNA-gel sample. Biotinylated lambda phage FISH probe (Enzo Life Sciences, Farmingdale, N.Y.) was diluted to 200 ng in hybridization buffer (Invitrogen Molecular Probes, Carlsbad, Calif.), and the sample was immersed in this solution. The solution was briefly heated to 80° C. for 3 minutes, incubated at 37° C. overnight, and finally washed 3×30 minutes in wash buffer (Invitrogen Molecular Probes, Carlsbad, Calif.). The sample was then incubated with Cy5-Streptavidin (1:200 in 1× PBS) and washed 2×30 minutes in PBS.

Polymer (e.g., Hydrogel) Expansion and Microscopic Detection

The hydrogel was expanded approximately twofold from its original size by washes in 1× PBS during DNA labelling. (To expand further, the sample could be exchanged into a buffer with lower salt content, such as 0.1× PBS). The sample was imaged in 1× PBS using an Andor spinning disk (CSU-X1 Yokogawa, Tokyo, Japan) confocal system with a 40× 1.15 NA water objective on a Nikon TI-E microscope body. Cy5-Streptavidin was excited with a 640 nm laser with a 685/40 emission filter.

### Results

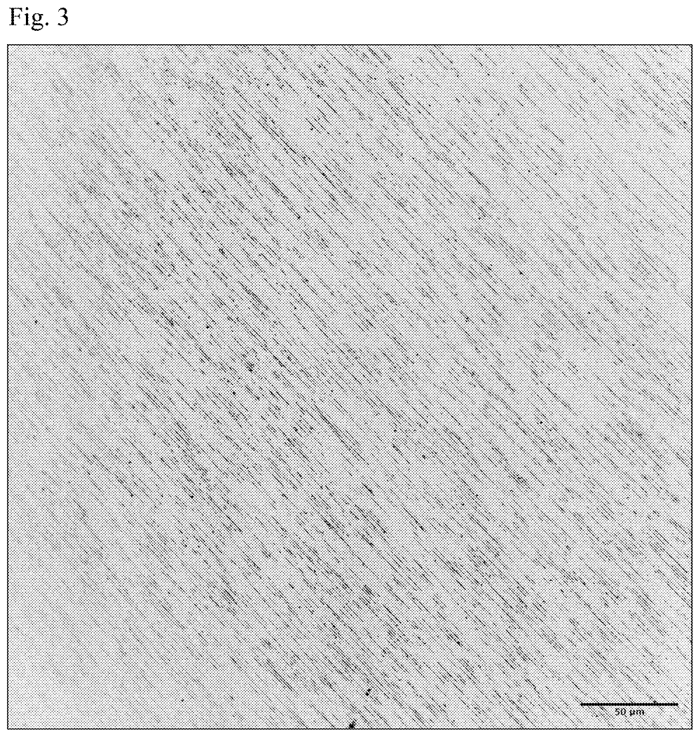

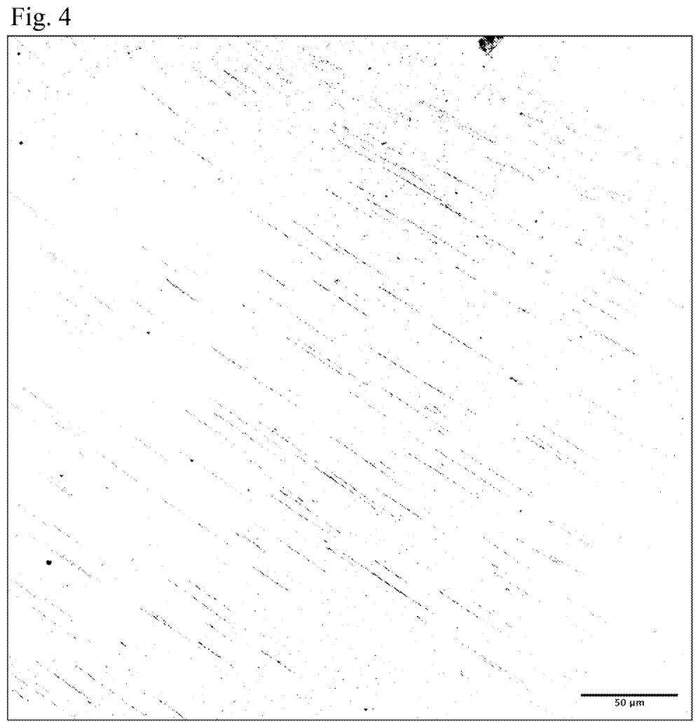

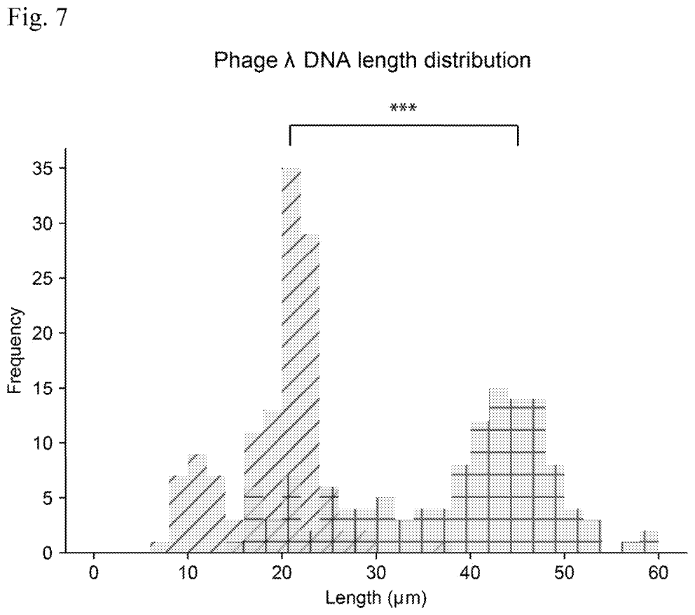

Lambda phage DNA was successfully prepared and embedded according to a method of the invention (FIGS. 3-4). Polymer-embedded linearized polynucleotide fragments were detectable using FISH probes and fluorescent microscopy both before (FIG. 3) and after expansion (FIG. 4) of the polymer in which the polynucleotides were embedded. Fragments remained linearized following expansion (compare FIGS. 3 and 4). Polymer expansion not only increased the three-dimensional space between embedded polynucleotide fragments, but also lengthened each fragment and increased the distribution of fragment lengths (FIG. 7; before expansion: μ=19.4, σ=5.0, n=126; after expansion: μ=39.2, σ=12.4, n=134). Fragment lengths were measured by manual annotation in FIJI image processing software. A line profile was fitted to each fragment in each microscopic image and the number of pixels spanned by the fragment was measured. Pixels were then converted into physical distances.

### Example 2

### Enzymatic Detection of Lambda Phage DNA with Polymerase

Enzymatic labelling with a polymerase, rather than labelling by hybridization, was performed in order to demonstrate how hydrogel embedding, unlike the solid phase, permits facile enzymatic analysis of linearized DNA.

### Materials and Methods

The steps of functionalization through hydrogel conversion were performed as described in Example 1. Following overnight NaOH incubation, the sample was washed 2×5 minutes in 1× PBS, then immediately exchanged into a primer-extension reaction (25 μM random hexamers (Thermaisher Scientific, Waltham, Mass.), 100 μM of each of dATP, dTTP, dGTP (New England Biolabs, Ipswich, Mass.), 40 μM biotin dCTP (ThermoFisher Scientific, Waltham, Mass.), 1:50 Klenow Fragment (3′→5′ exo-) (New England Biolabs, Ipswich, Mass.) in 1× NEBuffer 2). The reaction was incubated at 37° C. for one hour, then washed 3×5 minutes in PBS. Newly synthesized DNA was detected by a mouse anti-biotin antibody (1:200 in 1× PBS for 30 minutes, Abcam; ab201341) amplified by an Alexa-Fluor 488 Goat anti-mouse secondary (1:200 in 1× PBS, Thermo isher Scientific, Waltham, Maas.). Hydrogel expansion and microscopic detection were then performed as described in Example 1, with the exception that Alexa 488 was excited with a 488 nm laser with a 525/40 emission filter.

### Results

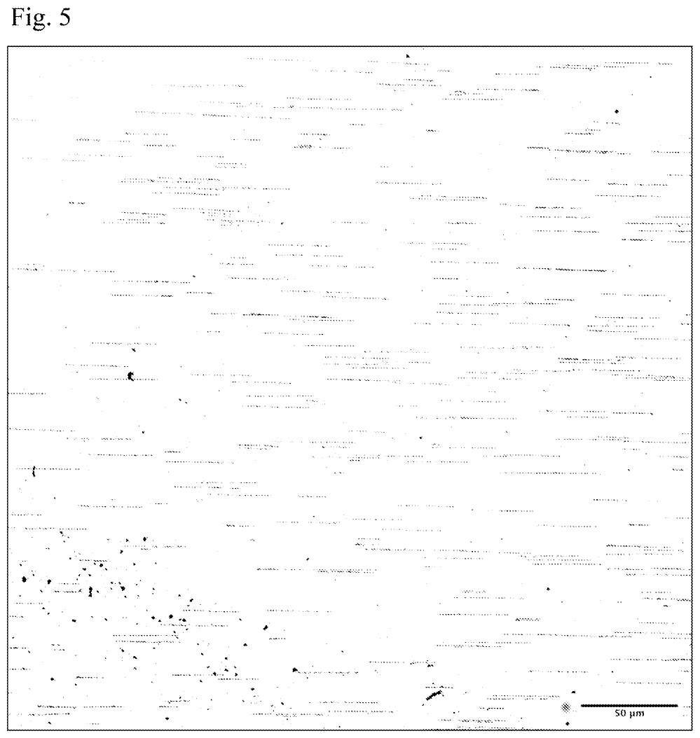

Polymerase-directed enzymatic labeling was successfully performed on hydrogel-embedded linearized lambda phage DNA fragments, and labelled DNA was successfully detected using fluorescence microscopy (FIG. 5).

### Example 3

### Enzymatic Detection of Lambda Phage DNA with Terminal Transferase

Enzymatic labelling with a terminal transferase was performed to demonstrate that the enzymatic labelling described in Example 2 is not a special case and that multiple types of enzymatic labelling may be performed on samples expanded according to methods of the invention.

### Materials and Methods

The steps of functionalization through hydrogel conversion were performed as described in Example 1. Following overnight NaOH incubation, the sample was washed 2×5 minutes in 1× PBS, then immediately exchanged into a primer-extension reaction (25 μM random hexamers (ThermoFisher Scientific, Waltham, Mass.), 100 μM dNTPs (New England Biolabs, Ipswich, Mass.), 1:50 Klenow Fragment (3′→5′ exo-) (New England Biolabs, Ipswich, Mass.) in 1× NEBuffer 2). The reaction was incubated at 37° C. for one hour, then washed 3×5 minutes in PBS. The sample was then exchanged into an end-tailing reaction (100 μM biotin dCTP (ThermoFisher Scientific, Waltham, Mass.), 1:20 terminal transferase (New England Biolabs, Ipswich, Mass.), 0.25 mM CoCl₂ in 1× Terminal Transferase Reaction Buffer (New England Biolabs, Ipswich, Mass.). This reaction was incubated at 37° C. for one hour, then washed 3×5 minutes in PBS. The reaction was then detected as described in Example 2.

### Results

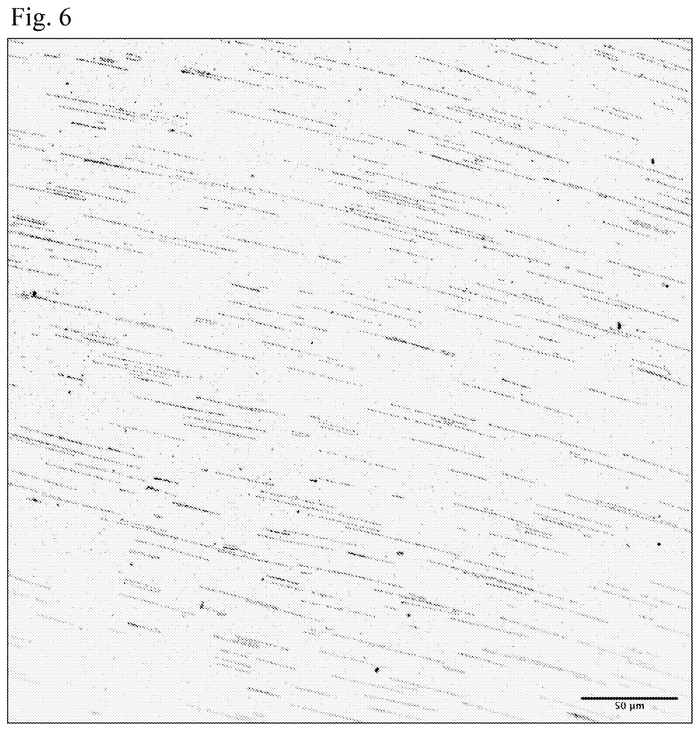

Terminal transferase-directed enzymatic labeling was successfully performed on hydrogel-embedded linearized lambda phage DNA fragments, and labelled DNA was successfully detected using fluorescence microscopy (FIG. 6).

### Example 4

### Detection, Mapping, and Cataloguing Spatial Structure and Sequence and Structural Features of Linearized Polynucleotides

Analyses of sequence and structural features are performed as follows:
1. A sample is prepared according to primer extension and terminal transferase methods described in Example 3 and elsewhere herein
2. Following terminal transferase treatment, tailed primers are circularized by Circligase enzyme and amplified by rolling circle amplification as described [U.S. Pat. No. 10,059,990; Lee, J., et al. Science 343: 1360-1363 (2014)].
3. Amplified DNA is then sequenced as described in [U.S. Pat. No. 10,059,990; Lee, J., et al. Science 343: 1360-1363 (2014)].
4. Distances between amplified DNA fragments are extracted from sequencing images and converted into genomic distances (e.g., 0.33 nm per base for elongated DNA, which is then multiplied by the expansion factor).
5. Measured sequence information of fragments is combined with measured genomic distances between fragments to yield relative genomic distances between measured sequences.
6. That information is subsequently used for one of more of: determining spatial structure of the linearized polynucleotides, determining sequence and structural features of the linearized polynucleotides, assembling genomes, and identifying structural variations in gene sequence.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of determining a structure and sequence of a polynucleotide, comprising:
(a) modifying the polynucleotide with a bi-functional cross-linker molecule;
(b) linearizing the polynucleotide;
(c) immobilizing the linearized polynucleotide;
(d) embedding the polynucleotide in a polymer material;
(e) fragmenting the embedded polynucleotide;
(f) physically expanding the polynucleotide fragments;
(g) detecting spatial position and sequences of the expanded polynucleotide fragments; and
(h) determining a structure and sequence of the polynucleotide.

2. The method of claim 1, wherein the modifying comprises functionalizing the polynucleotide, and optionally, a means for the functionalizing comprises: conjugating the polynucleotide to the bi-functional cross-linker molecule, wherein the bi-functional cross-linker molecule comprises at least one polynucleotide-reactive group and at least one material-reactive moiety.

3. (canceled)

4. The method of claim 2, wherein the at least one polynucleotide-reactive group comprises a DNA binding domain and the at least one material-reactive moiety comprises a polymerizable domain.

5-11. (canceled)

12. The method of claim 1, wherein the method is performed on a plurality of the polynucleotide.

13. The method of claim 12, wherein functionalizing the plurality of polynucleotides comprises conjugating the polynucleotides with two or more different bi-functional cross-linkers.

14-18. (canceled)

19. The method of claim 1, wherein the linearized polynucleotide is immobilized on a solid support.

20-21. (canceled)

22. The method of claim 1, wherein the polymer material comprises a swellable polymer material, or the polymer material comprises a non-swellable polymer material capable of conversion to a swellable polymer material and the method further comprises converting the non-swellable polymer material into a swellable polymer material.

23-24. (canceled)

25. The method of claim 22, wherein the method further comprises converting the non-swellable polymer material into a swellable polymer material prior to the physically expanding of the polynucleotide fragments.

26-28. (canceled)

29. The method of claim 19, further comprising cleaving the polymer material comprising the embedded polynucleotide from the solid support.

30-31. (canceled)

32. The method of claim 1, further comprising double-stranded denaturing the polynucleotide fragments embedded in the polymer material prior to the physical expansion of the polynucleotide fragments, wherein the double-stranded denaturing of the nucleotide fragments generates single-stranded polynucleotide fragments.

33. The method of claim 1, wherein a means of the physically expanding the polynucleotide fragments comprises expanding the polymer material in which the polynucleotide fragments are embedded, wherein the expansion of the polymer material expands the polynucleotide fragments isotropically in at least a linear manner within the polymer material.

34-37. (canceled)

38. The method of claim 1, further comprising passivating the expanded polymer.

39. The method of claim 1, wherein the detecting comprises one or both of imaging and sequencing the polynucleotide fragments.

40-47. (canceled)

48. The method of claim 1, further comprising detectably labeling the polynucleotide fragments.

49-53. (canceled)

54. The method of claim 39, wherein a means for the sequencing comprises:
(a) hybridizing one or more primer molecules to the polynucleotide fragments;
(b) amplifying the polynucleotide fragments; and
(c) determining sequences of the amplified polynucleotide fragments.

55-58. (canceled)

59. The method of claim 1, further comprising classifying the detected spatial positions and sequences of the expanded polynucleotide fragments into one or more contiguous polynucleotide molecule.

60-64. (canceled)

65. The method of claim 1, wherein the polynucleotide is obtained from a cell.

66. The method of claim 65, wherein the cell is obtained from a subject.

67. The method of claim 65, wherein the subject is a mammal, optionally is a human.

68. The method of claim 65, wherein the cell is a cultured cell.
